SEQUENCE LISTING (10 sequences)

SEQ ID NO: 1 - DNA, 91 bp, Bos taurus
gcatcgtggaagagtgttgcttccgaagctgcgacctggccctgctggagacttactgtgccacccccgccaagtccgagagggatgtgtc

SEQ ID NO: 2 - DNA, 84 bp, Bos taurus
gcgagacggtgcaggtggggcgctgtcgcctgcttctggcgcctcgtccccgcccggacggcgcggtcaataacccgcctcccg

SEQ ID NO: 3 - DNA, 584 bp, Bos taurus
gtgctgctgcaggtgttcctggaatgctgcatttactgggtaggattcgcttttcgaaatcctccagggacacagcccattgcgagaagtgaggtgttcaggtactccctgcagaagctggcatacacggtgtcgagaaccggacggcacgtgctgggagagcgccgccagcgggcccccaactgaggccccagccccctcccctgggcggccgtgtcaccaggtgctcctgtgccattccaccagcatgggagccagcgccgcgcaggaatggggcgtcccctgtgccctctcgccagaggagcacttgccaaggtcagtgaggggctggtaggcccctggagaagcagcaccgacaatgacgacaacaagagatcccttccccacccctctgcacccctcccactgcgggtggcctagagggaggaacggggcttagggggagcagactcctgagatctgggcacaggcaacgcattcagatctggaccaagtcgggacagcaccatcccagccccgcagccatggccatgccagcaggccgcaccacagagatccagacaactacaccagccagcagaatg

SEQ ID NO: 4 - DNA, 584 bp, Bos taurus
gtgctgctgcaggtgttcctggaatgctgcatttactgggtaggattcgcttttcgaaatcctccagggacacagcccattgcgagaagtgaggtgttcaggtactccctgcagaagctggcatacacggtgtcgagaaccggacggcacgtgctgggagagcgccgccagcgggcccccaactgaggccccagccccctcccctgggcggccgtgtcaccaggtgctcctgtgccattccaccagcatgggagccagcgccgcgcaggaatggggcgtcccctgtgccctctcgccagaggagcacttgccaaggtcagtgaggggctggtaggcccctggagaagcagcaccgacaatgacgacaacaagagatcccttccccacccctttgcacccctcccactgcgggtgtcctagagggaggaacggggcttagggggagcagactcctgagatctgggcacaggcaacgcattcagatctggaccaagtcgggacagcaccatcccagccccgcagccatggccatgccagcaggccgcaccacagagatccagacaactacaccagccagcagaatg

SEQ ID NO: 5 - DNA, 21 bp, Artificial Sequence
  PCR primer
gcatcgtggaagagtgttgct

SEQ ID NO: 6 - DNA, 21 bp, Artificial Sequence
  PCR primer
caagtccgagagggatgtgtc

SEQ ID NO: 7 - DNA, 16 bp, Artificial Sequence
  PCR primer
gcgagacggtgcaggt

SEQ ID NO: 8 - DNA, 19 bp, Artificial Sequence
  PCR primer
ctgaataacccgcctgggc

SEQ ID NO: 9 - DNA, 22 bp, Artificial Sequence
  PCR primer
gtgctgctgcaggtgttcctgg

SEQ ID NO: 10 - DNA, 26 bp, Artificial Sequence
  PCR primer
agacaactacaccagccagcagaatg

(19) United States
(12) Patent Application Publication
(10) Pub. No.: US 2010/0291576 A1
(43) Pub. Date: Nov. 18, 2010

(54) IMPRINTED GENES AS EPIGENETIC MARKERS FOR USE IN CLONING AND REGENERATIVE CELL PROCEDURES

(76) Inventors: Zhongde Wang, Sioux Falls, SD (US); James M. Robl, Canton, SD (US)

Correspondence Address: CLARK & ELBING LLP, 101 FEDERAL STREET, BOSTON, MA, 02110, US

(21) Appl. No.: 12/782,245

(22) Filed: May 18, 2010

Related U.S. Application Data
(60) Provisional application No. 61/179,130, filed on May 18, 2009.

(51) Int. Cl. C12Q 1/68

(52) U.S. Cl. 435/6

(57) ABSTRACT

In general, the invention features methods of identifying and/or preparing a mammalian cell or a cell population (e.g., bovine or porcine cells) for use in cloning procedures or regenerative cell procedures using expression of one or more of the epigenetic markers, IGF2, p57, and NNAT.

### CROSS-REFERENCE TO RELATED APPLICATION

This application claims the benefit of U.S. Provisional Patent Application No. 61/179,130, filed on May 18, 2009, herein incorporated by reference.

### FIELD OF THE INVENTION

The present invention relates to the field of genetic engineering.

### BACKGROUND OF THE INVENTION

Mammalian cloning procedures are generally inefficient as a number of clones are lost and do not result in the successful generation of viable offspring. In addition, cloned mammalian fetuses from diverse species often demonstrate similar undesirable phenotypes including enlarged fetuses and placentamegaly. We hypothesize that cloning inefficiency and the observed fetal phenotypes are attributed in part to epigenetic errors in the expression of imprinted genes.

Imprinted genes are mono-allelically expressed genes. The expression pattern of each imprinted gene is not random, but is determined by the parental genotype. For example, maternally-imprinted genes are only expressed from the paternal genome, while paternally-imprinted genes are only expressed in the maternal genome. Epigenetic markers for genomic imprinting are differentially established in the parental alleles in the germ cells during gametogenesis. These epigenetic markers are inherited and maintained in somatic tissues during embryogenesis by the offspring. These markers are erased and reset in the germ cells of the offspring in a sex-specific manner to establish the new parental-specific modifications for the next generation.

It is well known that cloned animals, even among different mammalian species, share some typical phenotypes, such as enlarged fetuses and placentamegaly. Aberrant imprinted gene expression is thought to contribute to these observed phenotypes, as well as to the decreased efficiency of cloning procedures. This hypothesis is supported by data from gene expression and chromatin modification studies which demonstrate that epigenetic errors in clones can be genome-wide and random. In addition, the importance of imprinted genes in regulating embryogenesis, both for the embryonic lineage and the trophoblast lineage, also supports a role for these genes in the successful development of cloned mammals. Perturbation of imprinted genes in both human and animal models has also been shown to cause abnormal fetal development.

Further evidence of the importance of imprinted genes on the outcome of cloning procedures is suggested by imprinting-related human pathologies, such as hydropic pregnancies caused by loss of imprinting of the p57 and/or Lit1 genes, which have a phenotype similar to the hydrop phenotypes observed in mammalian clones. In addition, many imprinted genes are abnormally regulated in cloned mammals from all species where clones have been produced and examined, which suggests that imprinted genes are particularly vulnerable to alteration during mammalian cloning. For example, the imprinted genes Lit1, Nap1L5, Zim2, and Mash2 have been demonstrated to have a loss of mono-allelic gene expression following cloning. Lastly, as imprinting errors can only be erased during the gametogenesis process, and cloned cells do not undergo gametogenesis in most cloning processes, a clone will inevitably suffer from such epigenetic errors. Such epigenetic errors will likely occur either in the donor cell before cloning or during the cloning process.

### SUMMARY OF THE INVENTION

We have discovered that there are particular sets of epigenetic errors that, different from other random errors, commonly occur in clones. Such common epigenetic errors may not only be responsible for the typical cloning phenotypes observed among various mammalian species, but may also contribute to clone loss during cloning procedures. Identification of a set of epigenetic genes that contribute to clone loss during cloning procedures allows for the development of strategies to avoid or even correct such misregulated genes in clones or allows for the selection of cells that do not express the misregulated genes. The selective removal of cells that express misregulated genes in cloning procedures would greatly improve cloning efficiency and the efficiency of generating cell lines for regenerative cell therapy.

By evaluating the expression of imprinted genes in cell lines used to generate cloned embryos and mammals, and statistical analysis of the gathered data, we have discovered three imprinted genes (IGF2, p57, and NNAT) that may be used to accurately predict the outcome of later cloning procedures.

Accordingly, in a first aspect, the invention features a method of identifying a mammalian cell or a cell population (e.g., bovine, porcine, ovine, or caprine cells) for use in cloning procedures (e.g., transfer of a donor cell, donor nucleus, or a donor chromatin mass, or permeabilized cell transfer procedures) or regenerative cell procedures (e.g., cellular reprogramming, generation of regenerative cell cultures, and induced pluripotent stem cell technology, e.g., using human cells). The method includes the steps of measuring the expression level of insulin-like growth factor-2 (IGF2) in the cell, measuring the expression level of p57 in the cell, and determining the ratio of the expression level of IGF2 to p57 in the cell, whereby a cell having an IGF2 to p57 ratio of less than or equal to 9 (e.g., less than 8, 7, 6, 5, 4, 3, or 2) is identified for use in a cloning procedure or regenerative cell procedure.

The invention further provides a method for identifying a mammalian cell for use in a cloning procedure or a regenerative cell procedure that includes the steps of determining whether the cell has mono-allelic expression of a neuronatin (NNAT) gene, whereby mono-allelic expression of a NNAT gene identifies a cell for use in a cloning procedure or a regenerative cell procedure.

Also provided by the present invention is a method for identifying a mammalian cell for use in a cloning procedure or a regenerative cell procedure that includes the steps of measuring the expression level of IFG2 and p57 in the cell and determining the ratio of the expression level of IGF2 to p57 in the cell, and determining whether the cell has mono-allelic expression of a NNAT gene, whereby an IGF2 to p57 ratio of less than or equal to 9 (e.g., less than 8, 7, 6, 5, 4, 3, or 2) and mono-allelic expression of a NNAT gene identifies a cell for use in a cloning procedure or a regenerative cell procedure.

The above methods provide for the identification of a mammalian cell that has at least a 30% (e.g., at least a 35%, 40%, 45%, 50%, 55%, 60%, 65%, 70%, 75%, 80%, 85%, 90%, 95%, 99%, or even 100%) probability of resulting in the generation of a viable embryo, viable offspring, or a genetically-stable regenerative cell culture.

The invention further provides a method of preparing a mammalian cell population for use in cloning procedures or regenerative cell procedures that includes the steps of measuring the expression level of IGF2 in one or more (for example, each) cell of the population, measuring the expression level of p57 in one or more (for example, each) cell in the population, determining the ratio of the expression level of IGF2 to p57 for one or more (for example, each) cell in the population, and selectively removing one or more (for example, each) cell from the population that has an IFG2 to p57 ratio of greater 2 (e.g., greater than 3, 4, 5, 6, 7, 8, 9, or 10).

Additional methods provided by the invention include methods for preparing a mammalian cell population for use in cloning or regenerative cell procedures that include the steps of determining whether one or more (for example, each) cell in the population has bi-allelic expression of a NNAT gene and selectively removing one or more (for example, each) cell from the population that have bi-allelic expression of a NNAT gene.

The invention further provides methods of preparing a mammalian cell population for use in cloning procedures or regenerative cell procedures including the steps of measuring the expression level of IGF2 and p57 in one or more (for example, each) cell of the population and determining the ratio of the expression of IGF2 to p57, determining whether one or more (for example, each) cell of the population has bi-allelic expression of a NNAT gene, and selectively removing one or more (for example, each) cell from the population that has an IGF2 to p57 ratio of greater than 2 (e.g., greater than 3, 4, 5, 6, 7, 8, 9, or 10) and has bi-allelic expression of a NNAT gene.

In each of the above methods, the selective removal of one or more (for example, each) cell having an IGF2 to p57 ratio of greater than 2 (e.g., greater than 3, 4, 5, 6, 7, 8, 9, or 10) and/or bi-allelic expression of a NNAT gene results in a population of cells that have at least a 30% (e.g., 35%, 40%, 45%, 50%, 55%, 60%, 65%, 70%, 75%, 80%, 85%, 90%, 95%, 99%, or even 100%) probability of resulting in the generation of a viable embryo, a viable offspring, or a genetically-stable cell culture following cloning procedures or regenerative cell procedures.

In each of the above methods, the expression levels of IGF2 and p57 may be determined by measuring the levels of mRNA encoding IGF2 and/or p57 (e.g., by reverse-transcriptase polymerase chain reaction (RT-PCR) or real time RT-PCR) or by measuring the IGF2 and/or p57 protein levels. The mono-allelic or bi-allelic expression of a NNAT gene, in any of the above methods, may also be determined by measuring the levels of mRNAs encoding a NNAT protein (e.g., RT-PCR or real time RT-PCR) or by measuring the level of a NNAT protein. The mono-allelic or bi-allelic expression of a NNAT gene, in each of the above methods, may also be measured by determining whether mRNA is transcribed from both alleles of a NNAT gene (e.g., by measuring the expression of different polymorphic sequences present in the two alleles of a NNAT gene using RT-PCR).

In each of the above methods, the resulting mammalian cells or mammalian cell cultures may be used in cloning procedures. Such cloning procedures may include transfer of a donor cell, donor nucleus, or donor chromatin mass, or a permeabilized cell to an enucleated oocyte and may also include as a step transfer of DNA (e.g., any heterologous and/or homologous DNA, such as insertion of a fragmented mammalian chromosome or a mammalian artificial chromosome into a cell). The cells identified by the invention or the cell lines provided by the invention may be used to generate an embryo to be inserted into a maternal host, and following gestation, may result in the generation of a live mammal (e.g., bovine, porcine, ovine, or caprine). The methods may also include any number of cloning steps (e.g., serial cloning) and the same or different methods may be used at each step.

The above methods may also generate mammalian cells or mammalian cell cultures that may be used in regenerative cell procedures. Such procedures include the growth and expansion of a primary cell, an embryonic stem cell, an undifferentiated cell, a cell from a blastocyst, a cell having a gene expression profile or phenotype similar to an undifferentiated cell or an embryonic stem cell, a reprogrammed cell, an induced pluripotent stem cell, or any of these cells following genetic modification. Embryonic stem cells, when expanded in culture, demonstrate heterogeneity in the expression of imprinted genes. The present methods allow for the identification of mammalian cells (e.g., human cells) that may be propagated in culture to yield a cell population that has decreased genetic heterogeneity in the expression of imprinted genes. The cells identified by the methods of the invention may be used in different regenerative cell procedures to yield genetically-modified cells (e.g., transfer of DNA or transgene expression of a wild-type allele of a mutated, dysfunctional, or nonfunctional gene) for cell replacement therapy. In addition, the present methods may be used to identify induced pluripotent stem (IPS) cells that may be propagated in culture to yield a genetically-stable cell population or may be successfully used in cloning procedures. The cells produced by the methods of the invention may also be used in the cloning and regenerative cell method of reprogramming. In reprogramming, a cell may be permeabilized and incubated with an interphase cell lysate to alter the expression of one or more genes (e.g., at least 2, 3, 4, 5, 6, 7, 8, 9, of 10 genes). The invention provides methods for the identification of reprogrammed cells that may be useful in cloning procedures and/or regenerative cell procedures.

Any of the above methods may further be used to identify a cell useful in a cloning procedure or a regenerative cloning procedure after one or more rounds of cloning (i.e., serial cloning). In addition, the mammalian cell or mammalian cell population of any of the above methods may be an ungulate cell (e.g., bovine, porcine, ovine, or caprine cell) for cloning procedures and any type of mammalian cell (including human cells) for regenerative cell procedures.

As used herein, by “bi-allelic expression” is meant the transcription of an mRNA from both alleles of a gene in the cell. Bi-allelic expression of a gene may be measured by determining the overall level of mRNA transcribed from both alleles of the gene. Bi-allelic expression of the gene may also be indicated by the relative level of the protein translated from mRNAs transcribed from both alleles of the gene. In determining the bi-allelic expression of a gene, the total level of mRNA encoding the gene or the total level of protein encoded by the gene in a cell may be compared to a control cell which is known to have bi-allelic or mono-allelic expression of the tested gene. The bi-allelic expression of a gene may also be determined by measuring the expression of different polymorphic sequences found in each of the two alleles of the gene. For example, the expression of two different alleles of the NKAT gene may be identified by the polymorphic sequences shown in FIG. 4.

By “cloning procedure” is meant any method which involves the artificial generation of an embryo, for example, the artificial transfer of a donor mammalian cell (for example, a permeabilized cell), donor nucleus, or donor chromatin mass from a mammalian cell into an enucleated oocyte. The embryo may or may not be introduced into a maternal host. The cloning procedures may include steps of genetically modifying an embryo (or cells used to produce the embryo) prior to implantation of the embryo into a maternal host including, but not limited to, inserting DNA (e.g., heterologous or homologous DNA, such as a fragmented mammalian chromosome fragment or an artificial chromosome carrying a xenogenous or endogenous gene) into a one-cell embryo, oocyte, or cell used to generate an embryo, as well as incubating a permeabilized cell in a reprogramming extract, wherein the cell may be further used to generate an embryo. The term “cloning procedure” also encompasses cloning methods that require multiple rounds of cloning (i.e., serial cloning). In any of the above-referenced cloning procedures, the donor cell, embryo, or oocyte may be genetically-modified (e.g., introduction of a fragmented mammalian chromosome, artificial chromosome, or transgene, or deletion, mutation, or truncation of an endogenous gene).

By “embryo” or “embryonic” is meant a developing cell mass that has not implanted into the uterine membrane of a maternal host. Hence, the term “embryo” may refer to a fertilized oocyte; an oocyte containing transferred DNA (e.g., heterologous and/or homologous DNA), or donor cell such as a reprogrammed cell or permeabilized cell; a pre-blastocyst stage developing cell mass; or any other developing cell mass that is at a stage of development prior to implantation into the uterine membrane of a maternal host and prior to formation of a genital ridge. An embryo may represent multiple stages of cell development. For example, a one-cell embryo can be referred to as a zygote; a solid spherical mass of cells resulting from a cleaved embryo can be referred to as a morula, and an embryo having a blastocoel can be referred to as a blastocyst. An “embryonic cell” is a cell isolated from or contained in an embryo.

By “enucleated oocyte” is an oocyte that has had its nucleus removed. Methods for the purification and removal of the nucleus from an oocyte are known in the art and include both physical and chemical methods.

By “expression level” is the meant the level of an mRNA expressed from one or both alleles of a gene or the level of a protein expressed from the mRNA produced by one or both alleles of a gene. The level of expression of an mRNA or protein may be compared to the level of expression of a different mRNA or protein in the cell (e.g., level of expression of IGF2 compared to the expression of p57 in a cell). The level of the expression of an mRNA relative to the level of expression of a second mRNA may be compared using the technique of real-time RT-PCR. The method of RT-PCR provides a value for the expression of an mRNA encoding one gene relative to the level of expression of an mRNA encoding a second gene or a standard curve. The expression level of an mRNA in an experimental cell may also be compared to the level of expression of the same mRNA in a control cell (e.g., a fetal fibroblast, a differentiated adult fibroblast, or a germ cell).

The level of expression of a protein may also be compared. The level of expression of a protein may be measured using a number of techniques that utilize, for detection, an antibody specific for the protein of interest. Such methods include, but are not limited to, protein array technology, Western blotting, fluorescence-assisted cell sorting (FACS) using fluorescently-labeled antibodies, and enzyme-linked immunoabsorbant assay (ELISA). The level of expression of a specific protein in a cell population or cell may be compared to the level of expression in a control cell (e.g., a fetal fibroblast, a differentiated adult fibroblast, or a germ cell) or control cell population. In addition, the level of expression in a cell may be compared to the expression of a control gene within the same cell (e.g., β-actin).

By “genetically-stable regenerative cell line” is meant a cell that maintains its pattern of imprinted gene expression after cultivation (e.g., one or more cell divisions or population doublings, e.g., at least 1, 2, 3, 4, 5, 6, 7, 8, 9, 10, 15, 20, 25, 30, 50, 70, 100, 150, 200, 250, 300, 350, or 400 cell divisions or population doublings) in vitro. A cell derived from a blastocyst initially maintains its imprinted gene expression pattern, but following multiple rounds of cell division, the derived cells often demonstrate heterogeneity in their observed patterns of imprinted gene expression. Examples of genes that may show altered expression patterns following in vitro culture include, but are not limited to, one or more of Lit1, Nap1L5, Zim2, and Mash2. Heterogeneity in imprinted gene expression within a cell population is thought to contribute to the inconsistent phenotype for cultured regenerative cells.

By “imprinted gene” or “epigenetic marker” is meant a gene having alleles that are expressed in a parent-of-origin specific manner. The inheritance of expression of the alleles of an imprinted gene do not follow classical Mendelian inheritance and are expressed only from the allele of the gene inherited from the mother (e.g., H19 or CDK41C) or from the allele inherited from the father (e.g., IGF2). Imprinted genes are often regulated by cellular modification of the chromatin structure rather than by function of transcription activators or transcription inhibitors that increase or decrease gene transcription from each allele, respectively. Additional examples of imprinted genes include, but are not limited to, IGF2, p57, NNAT, H19, CDK41C, Lit1, Nap1L5, Zim2, and Mash2.

“Insulin-like growth factor-2” or “IGF2” is a member of the insulin family of polypeptide growth factors that is involved in development and growth. The IGF2 gene is an imprinted gene that is only expressed from the paternally inherited allele. The IGF2 mRNA sequence of human (NCBI Accession Nos. NM_{—}000612 and NM_{—}001007139), bovine (NCBI Accession No. BC126514), porcine (NCBI Accession No. NM_{—}213883), ovine (NCBI Accession No. NM_{—}001009311), and caprine IGF2 (DQ645739) are available on the NCBI website. The protein sequence for human (NCBI Accession No. AAA60088), bovine (NCBI Accession No. NP_{—}776512), porcine (NCBI Accession No. NP_{—}999048), ovine (NCBI Accession No. NP_{—}001009311), and caprine IGF2 (NCBI Accession No. ABG33777) are also available on the NCBI website.

By “interphase cell extract” is meant a cell extract prepared from a cell in interphase. An interphase cell extract induces chromatin decondensation and/or nuclear envelope formation. By “mitotic cell extract” is meant a cell extract from a mitotic cell that induces chromatin condensation and nuclear envelope breakdown.

By “live calving rate” is meant the percentage of implanted embryos that result in the production of viable offspring. A ≧2% live calving rate indicates that ≧2% of the total number of implanted embryos result in the production of a viable offspring. A ≧5% live calving rate indicates that ≧5% of the total number of implanted embryos result in the production of a viable offspring. The term “at least one single live cell” is meant that at least one single live offspring resulted from the cloning procedures.

By “mono-allelic expression” is meant the transcription of an mRNA from one allele of a gene in a cell. Mono-allelic expression of a gene may be measured by determining the overall level of mRNA encoding the protein of the expressed gene (i.e., the total level of mRNA transcribed) and comparing this level of mRNA to the total level of the same mRNA in a control cell having bi-allelic or mono-allelic expression of the same gene. The mono-allelic expression of a gene may also be determined by measuring the expression of different polymorphic sequences found in each of the two alleles of the gene. For example, the expression of two different alleles of the NNAT gene may be identified by the polymorphic sequences shown in FIG. 5.

“Neuronatin” or “NNAT” is a proteolipid that has been implicated for a role in the regulation of iron channels during brain development. NNAT plays a role in forming and maintaining the structure of the nervous system. NNAT is an imprinted gene that is expressed only from the paternal allele. The NNAT mRNA sequence for human (NCBI Accession Nos. NM_{—}005386 and NM_{—}181689), bovine (NCBI Accession Nos. NM_{—}178323 and BC103128), mouse (NCBI Accession No. BCO36984), rat (NCBI Accession No. BC127473), and porcine NNAT (NCBI Accession No. NM_{—}001122990) are available on the NCBI website. The protein sequence for human (NCBI Accession Nos. CAC00477, NP_{—}005377, and NP_{—}859017), bovine (NCBI Accession Nos. AA103129, NP_{—}847893, and AAR12962), mouse (NCBI Accession Nos. CAM16145, NP_{—}035053, and NP_{—}851291), rat (NCBI Accession Nos. NP_{—}859015 and NP_{—}446053), and porcine NNAT (NCBI Accession Nos. ABG72731 and NP_{—}001116462) are also available on the NCBI website.

“p57” is an imprinted gene that has been implicated as having a role in the human disorder, Beckwith-Wiedemann syndrome. The p57 mRNA sequence for human (NCBI Accession Nos. NM_{—}001122631, NM_{—}001122630, NM_{—}000076, BC039188, and EU570054), rat (NCBI Accession Nos. NM_{—}001033757, NM_{—}001033758, NM_{—}182735, and BC098646), mouse (NCBI Accession No. NM_{—}009876), bovine (NCBI Accession Nos. BC 123620, and NM_{—}001077903), and monkey (NCBI Accession No. XM_{—}001117302) are available on the NCBI website. The p57 protein sequence for human (NCBI Accession No. AAH67842, NP_{—}001116103, NP_{—}001116102, and NP_{—}000067), rat (NCBI Accession Nos. AAT84266 and NP_{—}877399), mouse (NCBI Accession Nos. NP_{—}034006 and AAH05412), bovine (NCBI Accession No. AAI23621), and monkey (NCBI Accession No. XP_{—}001117302) are also available on the NCBI website.

By “permeabilization” or “permeabilized” is meant the formation of pores in the plasma membrane or the partial or complete removal of the plasma membrane.

By “polymorphism” or “polymorphic sequence” is meant the nucleic acid sequence of an allele of a gene that differs from the sequence in the second allele of the same gene. A polymorphic sequence represents a difference in one, two, three, four, five, six, seven, eight, nine, or ten nucleotides from the sequence of the second allele of the same gene. Methods for measuring polymorphic sequences in a gene include, but are not limited to, RT-PCR, real time RT-PCR, and single-strand conformation polymorphism (SSCP) techniques.

By a “regenerative cell procedure” is meant a method or a step in a method of generating a population of primary cells, adult stem cells, embryonic stem cells, undifferentiated cells, or cells having a phenotype or a gene expression pattern similar to an embryonic stem cell, adult stem cell, or an undifferentiated cell. This term includes methods of propagating a primary cell, an adult stem cell, an embryonic stem cell, an undifferentiated cell, or a cell having a phenotype or a gene expression pattern similar to an embryonic stem cell, an adult stem cell, or an undifferentiated cell in vitro, and may also include the subsequent treatment (e.g., genetic modification or differentiation) of such cells. Regenerative cell procedures are used, for example, to yield cell cultures useful for research and for administration to subjects (e.g., humans) for therapy, such as replacement cell therapy.

Regenerative cell procedures also include techniques such as induced pluripotent stem cell technology, whereby a target cell (e.g., a fibroblast) is transduced or transfected with the genes for multiple, for example, three or four transcription factors, which confer on the target cell the phenotype or a pattern of cell expression similar to that of an embryonic stem cell, an adult stem cell, or an undifferentiated cell. Such transduced or transfected cells may be differentiated to a particular cell type, used in cloning procedures, used for research, or administered to a subject (e.g., a human) in need of replacement cell therapy.

Regenerative cell procedures also include the reprogramming of a target cell with a cell extract (e.g., extract from an interphase cell or a metaphase cell, such as a metaphase II oocyte). In one exemplary procedure, the target cell (e.g., a fibroblast) is first permeabilized and then incubated with a cell extract. The permeabilization and incubation of the target cell in cell extract results in an alteration in the expression of at least one gene (e.g., at least 2, 3, 4, 5, 6, 7, 8, 9, or 10 genes) in the target cell. The resulting cell may express an embryonic stem cell, an adult stem cell, or undifferentiated cell phenotype and may be further differentiated to a specific cell type, used in research, or used for regenerative cell therapy.

By “reprogramming media” is meant a solution that allows the removal of a factor from a cell, nucleus, or chromosome, or the addition of a factor from the solution to the cell, nucleus, or chromosome. Preferably, the addition or removal of a factor increases or decreases the level of expression of an mRNA or protein in the donor cell. In another embodiment, incubating a permeabilized cell in the reprogramming media alters a phenotype of the permeabilized cell relative to the phenotype of the donor cell. In yet another embodiment, incubating a permeabilized cell in the reprogramming media causes the permeabilized cell to gain or lose an activity relative to the donor cell.

Exemplary reprogramming media include solutions, such as buffers, that do not contain biological molecules such as proteins or nucleic acids. Such solutions are useful for the removal of one or more factors from a nucleus or chromosome. Other preferred reprogramming medias are extracts, such as cellular extracts from cell nuclei, cell cytoplasm, or a combination thereof Exemplary cell extracts include extracts from oocytes (e.g., mammalian, vertebrate, or invertebrate oocytes), male germ cells (mammalian, vertebrate, or invertebrate germ cells such as spermatogonia, spermatocyte, spermatid, or sperm), and stem cells (e.g., adult or embryonic stem cells). Yet other reprogramming media are solutions or extracts to which one or more naturally-occurring or recombinant factors (e.g., nucleic acids or proteins such as DNA methyltransferases, histone deacetylases, histones, protamines, nuclear lamins, transcription factors, activators, repressors, chromatin remodeling proteins, growth factors, interleukins, cytokines, or other hormones) have been added, or extracts from which one or more factors have been removed. Still other reprogramming media include solutions of detergent (e.g., 0.01% to 0.1%, 0.1% to 0.5%, or 0.5% to 2% ionic or non-ionic detergent such as one or more of the following detergents: SDS, Triton X-100, Triton X-114, CHAPS, Na-deoxycholate, n-octyl glucoside, Nonidet P40, IGEPAL, Tween 20, Tween 40, or Tween 80), salt (e.g., ˜0.1, 0.15, 0.25, 0.5, 0.75, 1, 1.5, or 2 M NaCl or KCl), polyamine (e.g., ˜1 μM, 10 μM, 100 μM, 1 mM or 10 mM spermine, spermidine, protamine, or poly-L-lysine), a protein kinase (e.g., cyclin-dependent kinase 1, protein kinase C, protein kinase A, MAP kinase, calcium/calmodulin-dependent kinase, CK1 casein kinase, or CK2 casein kinase), and/or a phosphatase inhibitor (e.g., ˜10 μM, 100 μM, 1 mM, 10 mM, 50 mM, 100 mM of one or more of the following inhibitors: Na-orthovanadate, Na-pyrophosphate, Na-fluoride, NIPP1, inhibitor 2, PNUTS, SDS22, AKAP 149, or ocadaic acid) or nuclioplasmin. In some embodiments, the reprogramming medium contains an anti-NuMA antibody. If desired, multiple reprogramming media may be used simultaneously or sequentially to reprogram a donor cell.

By “reprogrammed cell” is meant a cell that has been treated to alter the expression of one or more of its genes. Preferably, at least 1, 5, 10, 15, 20, 25, 50, 75, 100, 150, 200, 300, or more mRNA or protein molecules are expressed in the reprogrammed cell that are not expressed in the cell prior to reprogramming. In another preferred embodiment, the number of mRNA or protein molecules that are expressed in the reprogrammed cell, but not expressed in cell prior to reprogramming, is between 1 and 5, 5 and 10, 10 and 25, 25 and 50, 50 and 75, 75 and 100, 100 and 150, 150 and 200, or 200 and 300, inclusive. Preferably, at least 1, 5, 10, 15, 20, 25, 50, 75, 100, 150, 200, 300, or more mRNA or protein molecules are expressed in the cell prior to reprogramming that are not expressed in the reprogrammed cell. In yet another preferred embodiment, the number of mRNA or protein molecules that are expressed in the cell prior to reprogramming, but not expressed in the reprogrammed cell, is between 1 and 5, 5 and 10, 10 and 25, 25 and 50, 50 and 75, 75 and 100, 100 and 150, 150 and 200, or 200 and 300, inclusive. In still another preferred embodiment, these mRNA or protein molecules are expressed in the cell prior to programming and the reprogrammed cell, but the expression levels in the reprogrammed cells differ by at least 2, 5, 10, or 20-fold, as measured using standard assays (see, for example, Ausubel et al., supra). One example of a reprogrammed cell is an induced pluripotent stem (IPS) cell.

By “serial cloning” is meant two or more rounds of successive cloning. For example, a cell from an embryo, fetus, or adult may be genetically modified, and the cell or its nucleus may be inserted into an enucleated oocyte and implanted in a maternal host to generate a second embryo, fetus, or adult. The cells from the second embryo, fetus or adult may be used in future rounds of cloning. Each step in the cloning process includes the separation of a cell from the embryo, fetus, or adult mammal, subsequent treatment or genetic modification, and introduction of the cell or its nucleus into an enucleated oocyte to generate a further embryo. “G0” represents a cell prior to genetic modification or a cloning procedure (e.g., a primary cell). “G1” represents a cell from an embryo, fetus, or adult generated from a first cloning procedure. “G2” represents a cell that has undergone two rounds of cloning. “G3-5” indicates a cell that has undergone three to five rounds of serial cloning (e.g., “G3” is a cell resulting from three rounds of cloning, “G4” is a cell resulting from four rounds of cloning, and “G5” is a cell resulting from five rounds of cloning).

By “viable offspring” is meant an animal that survives ex utero. Preferably, the mammal is alive for at least one second, one minute, one hour, one day, one week, one month, six months, or one year from the time it exits the maternal host. The animal does not require the circulatory system of an in utero environment for survival.

### BRIEF DESCRIPTION OF THE DRAWINGS

FIG. 1 is data showing the statistical probability that each of a cell line having an IGF2/p57 expression ratio of less than 9 (IGF2/p57<9), a cell line having an IGF2/p57 ratio of greater than 9 (IGF2/p57>9), and a randomly selected cell line will result in production of a single bovine calf, a ≧2% live calving rate, or a ≧5% live calving rate at several steps in a serial cloning procedure. G0-1 represents cell lines that have not undergone a cloning step and cell lines derived from a first cloned fetus; G2 represents cell lines derived from a second cloned fetus (i.e., a cell that has undergone two cloning cycles); and G3-5 represents cell lines that have undergone three to five cloning cycles. Ninety cell lines were analyzed. For each cell line, an average of more than fifty embryos were generated and transferred to recipient females (i.e., more than 4,500 embryos were generated and transferred).

FIG. 2 is data showing the statistical probability that each of a cell line having mono-allelic expression of NNAT (NNAT<2), a cell line having bi-allelic expression of NNAT (NNAT>2), and a randomly selected cell line will result in production of a single live calf, a ≧2% live calving rate, or a ≧5% live calving rate at several steps in a serial cloning procedure. G0-1 represents cell lines that have not undergone a cloning step and cell lines derived from a first cloned fetus; G2 represents cell lines from a second cloned fetus (i.e., a cell that has undergone two cloning cycles); and G3-5 represents cell lines that have undergone three to five cloning cycles. Sixty-one cell lines were analyzed. For each cell line, an average of more than fifty embryos were generated and transferred to recipient females (i.e., a total of more than 3,050 embryos were generated and transferred).

FIG. 3 is data showing the statistical probability that each of a cell line having an IGF2/p57 expression ratio of less than 9 (IGF2/p57<9), a cell line having mono-allelic expression of NNAT (NNAT<2), a cell line having an IGF2/p57 expression ratio of less than 9 and mono-allelic expression of NNAT (“Double Selection”), a cell line having an IGF2/p57 expression ratio of greater than 9 (IGF2/p57>9) and bi-allelic expression of NNAT (NNAT≧2), and a randomly selected cell line will result in production of a single live calf, a ≧2% live calving rate, or a ≧5% live calving rate at several steps in a serial cloning procedure. G0-1 represents cell lines that have not undergone a cloning step and cell lines derived from a first cloned fetus; G2 represents cell lines from a second cloned fetus (i.e., a cell that has undergone two cloning cycles); and G3-5 represents cell lines that have undergone three to five cloning cycles. Sixty-three cell lines were analyzed. For each cell line, an average of more than 50 embryos were generated and transferred (i.e., a total of more than 3,150 embryos were generated and transferred).

FIG. 4A is a PCR product of bovine IGF2 (SEQ ID NO: 1). The primers used to amplify the cDNA sequence of IGF2 are indicated in bold. FIG. 4B is a PCR product of bovine p57 (SEQ ID NO: 2). The primers used to amplify the cDNA sequence of p57 are indicated in bold.

FIG. 5 is a PCR product of bovine NNAT (SEQ ID NOS: 3 and 4). The polymorphic differences in the sequences of the two alleles of NNAT are indicated in bold. The primer sequences used to amplify the NNAT nucleic acid sequence are shown in bold and underlined.

FIG. 6 is a DNA sequencing chromatogram showing the bi-allelic expression of NNAT (nucleotides 386 to 421 of SEQ ID NO: 3). The double peaks representing the different polymorphic sequences in the each allele of NNAT are indicated by two boxes (labeled as nucleotide numbers 321 and 344 in FIG. 6).

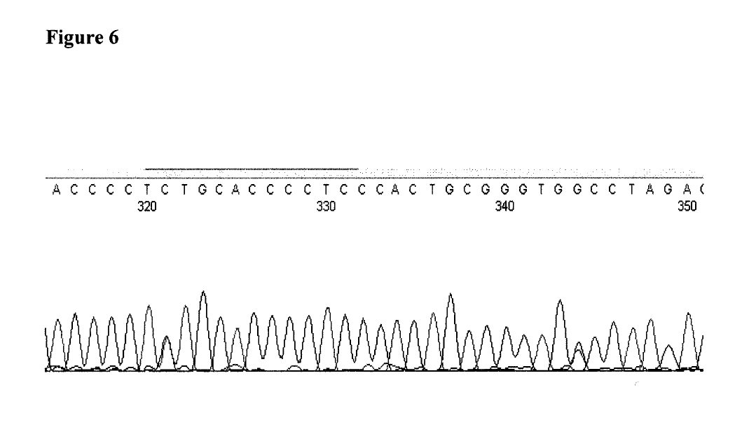

Other features and advantages of the invention will be apparent from the following detailed description and from the claims.

### DETAILED DESCRIPTION

We have developed methods for identifying cells and cell lines that are highly successful in cloning and regenerative cell procedures. Through statistical analysis of cloning data and gene expression data for each of 21 different imprinted genes, we have identified three epigenetic markers (IGF2, p57, and NNAT) that may be used to identify and select for cells useful for generating a viable embryo, a viable offspring, or a genetically-stable cell line.

### Methods for Identifying a Cell for Use in Cloning Procedures or Regenerative Cell Procedures

The present invention provides methods for identifying a mammalian cell or cell line useful in cloning procedures or regenerative cell procedures that require the measurement of the IGF2/p57 expression ratio and/or the allelic expression of a NNAT gene. Cells that may used in the present invention include cells from embryos, blastocysts, fetuses, young animals (such as calves), or adult animals. Cells that may be used include undifferentiated cells, embryonic stem cells, adult stem cells, fetal fibroblasts, or cells from the ectoderm, mesoderm, endoderm, or mesenchyme of an embryo. Additional cells that may be used in the invention include cells from an adult mammal including, but not limited to, fibroblasts, epithelial cells, neural cells, epidermal cells, keratinocytes, hematopoietic cells, melanocytes, chondrocytes, B-lymphocytes, T-lymphocytes, erythrocytes, macrophages, monocytes, placental, and muscle cells. Additional cells that may be used in the present invention include those from any organ, such as the bladder, brain, esophagus, fallopian tube, heart, intestines, gallbladder, kidney, liver, lung, ovaries, pancreas, prostate, spinal cord, spleen, stomach, testes, thymus, thyroid, trachea, ureter, urethra, and uterus.

Cells used in the methods of the invention may include cells from any mammal, including, but not limited to primates, mice, rats, dogs, cats, and ungulates. Ungulates include members of the orders Perissodactyla and Artiodactyla, such as any member of the genus Bos. Other preferred ungulates include sheep, big-horn sheep, goats, buffalos, antelopes, oxen, horses, donkeys, mule, deer, elk, caribou, water buffalo, camels, llama, alpaca, pigs, and elephants. Most preferably, the non-human mammal is a bovine (e.g., Bos taurus or Bos indicus). The use of human cells in the methods of the invention may be used for regenerative procedures. Any mammalian cell, for example, those preferred cells above, may also be used for regenerative cell procedures.

If a cell is derived from an embryo or a fetus (e.g., as part of a first cloning step or a serial cloning step), the cell may be isolated at any time during the gestation period. Bovine cells are desirably isolated at between 25 to 90 days of gestation, between 35 to 60 days of gestation, between 35 to 50 days, preferably between 35 to 45 days, more preferably between 38 to 43 days, and most preferably at about 40 days of gestation. Ovine cells are desirably isolated at between 25 to 150 days of gestation, between 30 to 100 days, preferably between 35 to 80 days, more preferably between 35 to 60 days, and most preferably at about 40 days of gestation. Equine cells are desirably isolated at between 25 to 300 days of gestation, between 30 to 100 days, preferably between 35 to 80 days, more preferably between 35 to 60 days, and most preferably at about 40 days of gestation. Porcine cells are desirably isolated at between 25 to 110 days of gestation, between 30 to 90 days, preferably between 30 to 70 days, more preferably between 30 to 50 days, and most preferably at about 35 days of gestation. Caprine cells are desirably isolated at between 25 to 150 days of gestation, between 30 to 100 days, preferably between 35 to 80 days, more preferably between 35 to 60 days, and most preferably at about 40 days of gestation. Primate cells are desirably isolated at between 25 to 150 days of gestation, between 30 to 100 days, preferably between 35 to 80 days, more preferably between 35 to 60 days, and most preferably at about 40 days of gestation. Rodent cells are desirably isolated at between 6 to 18 days of gestation, between 8 to 16 days, preferably between 10 to 16 days, more preferably between 12 to 16 days, and most preferably at about 14 days of gestation.

A mammalian cell or cell line for use in cloning procedures or regenerative cell procedures may be identified by measuring the IGF2/p57 expression ratio, the allelic expression of a NNAT gene, or the combination of the IGF2/p57 expression ratio and the allelic expression of a NNAT gene. Methods for the determination of the IGF2 and p57 expression levels, NNAT allelic expression levels, and the calculation of the IGF2/p57 ratio in mammalian cells are described below.

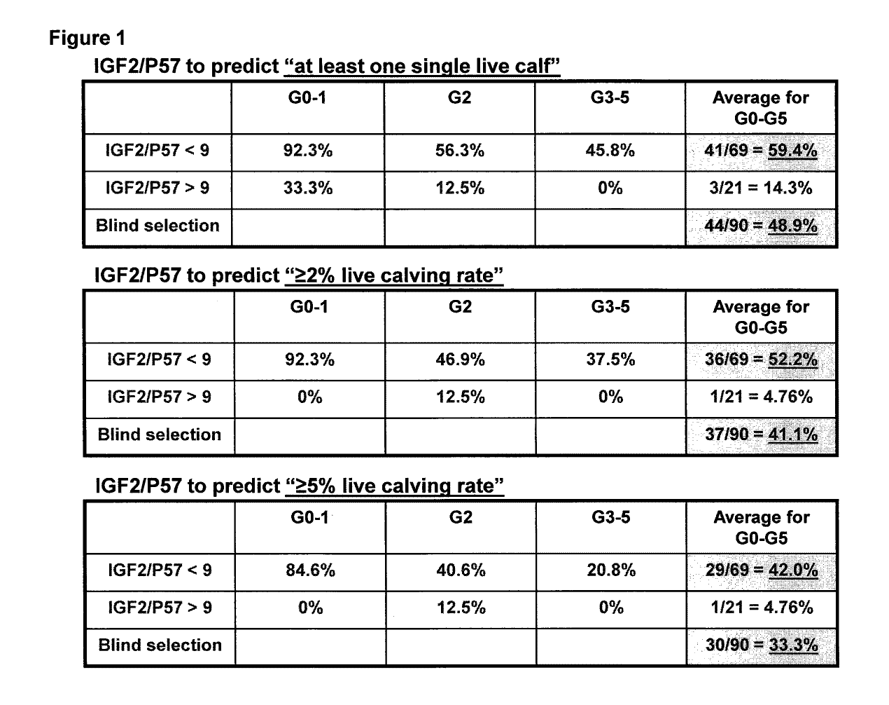

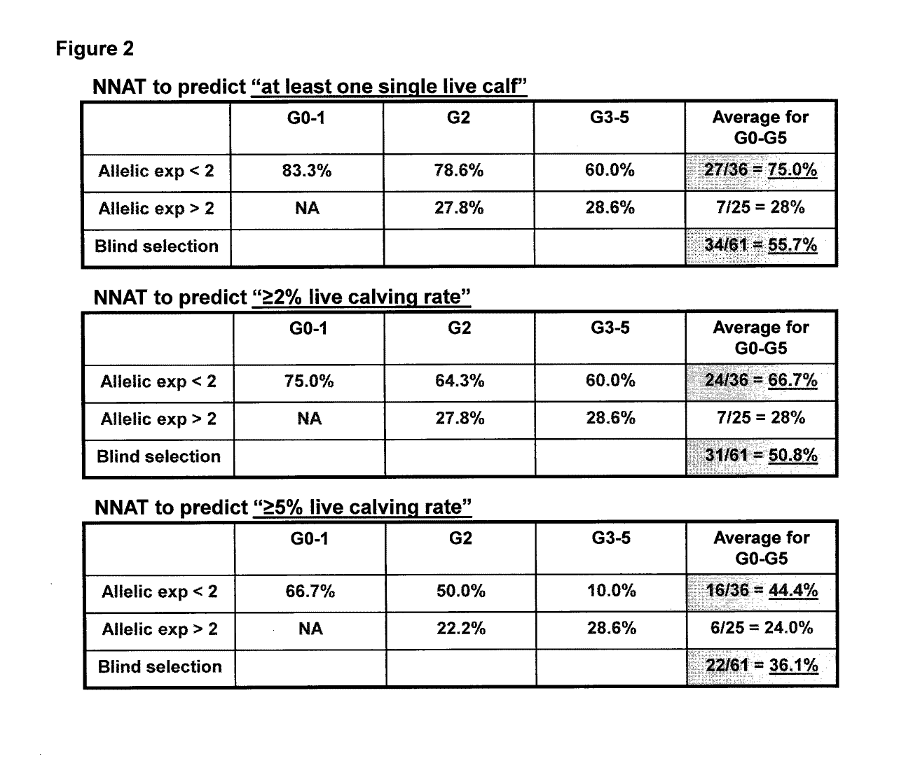

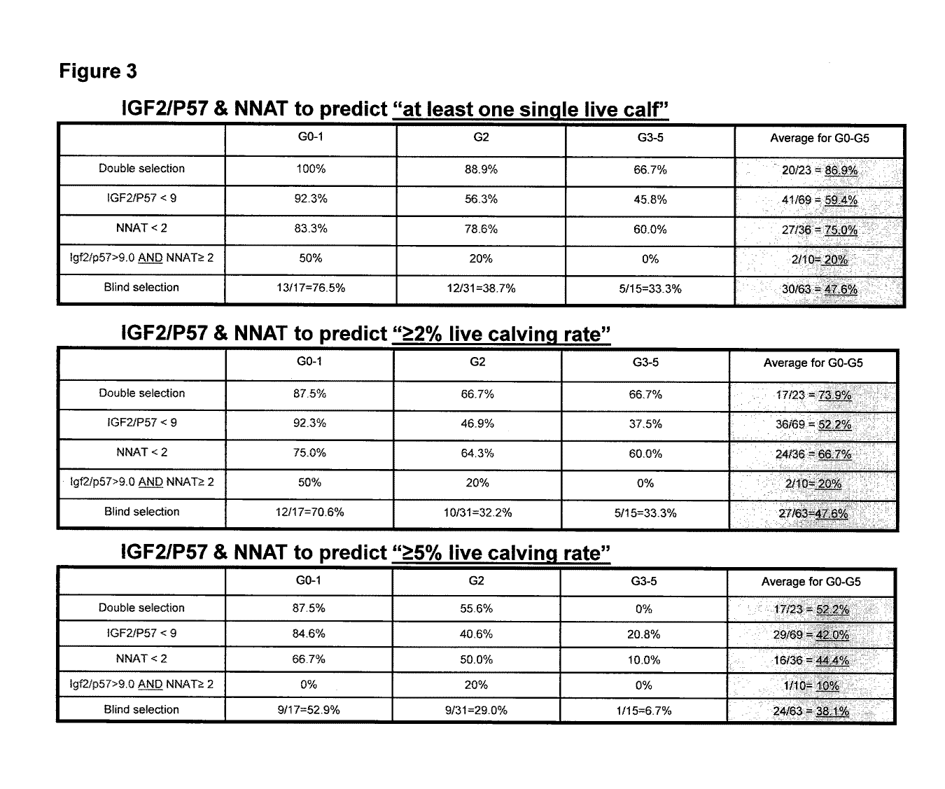

For bovines, an IGF2/p57 expression ratio of less than 9 was discovered to strongly correlate with the ability of a cell to generate a viable offspring in cloning procedures (FIG. 1). Mono-allelic expression of NNAT was also discovered to correlate strongly with the ability of a bovine cell to generate a viable bovine offspring following cloning procedures (FIG. 2). Successful generation of a viable bovine offspring was most highly correlated with cells that had both an IGF2/p57 expression ratio of less than 9 and mono-allelic expression of NNAT (FIG. 3).

Although an IGF2/p57 ratio of less than 9 was discovered to highly correlate with the ability of a bovine cell line to generate a viable offspring in cloning procedures, the optimal expression ratio of IGF2/p57 that may be used to indicate a successful cell for use in cloning procedures may differ for each mammalian species. For example, the ratio of IGF2/p57 expression may be less than 15, less than 14, less than 13, less than 12, less than 11, less than 10, less than 9, less than 8, less than 7, less than 6, less than 5, less than 4, less than 3, or less than 2. The exact IGF2/p57 expression ratio for each mammalian species may be determined through the measurement of the IGF2/p57 expression ratio for cell lines, measurement of the ability of each cell line to generate a viable cloned offspring, and the statistical analysis of the resulting data (as described in the Examples for bovine cell lines).

### Measurement of Mammalian IGF2 Expression Levels

Insulin-like growth factor-2 (IGF2) expression levels may be measured by determining the quantity of mRNA encoding IGF2 protein in a mammalian cell or cell line, or by determining the quantity of IGF2 protein expressed in a mammalian cell or cell line.

The level of mRNA encoding IGF2 protein may be measured using polymerase chain reaction (PCR)-based techniques, including real-time RT-PCR and RT-PCR. The specific primers used for each mammalian species may be designed from the IGF2 cDNA sequences from different mammalian species. Available IGF2 cDNA sequences include, but are not limited to, human (NCBI Accession Nos. NM_{—}000612, NM_{—}001007139, and NM_{—}001127598), rat (NCBI Accession No. NM_{—}031511), mouse (NCBI Accession Nos. NM_{—}010514, NM_{—}001122737, and NM_{—}019969), bovine (NCBI Accession No. BC126514), porcine (NCBI Accession No. NM_{—}213883), ovine (NCBI Accession No. NM_{—}001009311), and caprine IGF2 (DQ645739). Primer sequences used to amplify and quantitate the mRNAs encoding IGF2 in a mammalian cell may be designed using publicly available software on the NCBI website, as well as software programs available on several commercial websites, including the Invitrogen and Biosearch Technologies websites. An example of a set of specific primers used to amplify bovine IGF2 mRNA are described in the Examples. The level of IGF2 mRNA expression may be normalized to the level of expression of a control gene in the cell (e.g., β-actin).

The cDNA sequence of IGF2 for mammalian species not yet cloned may be obtained using standard techniques in molecular biology. For example, due to the high degree of conservation of the protein sequence of IGF2 between mammalian species, degenerative primers based on the cDNA or protein sequence of IGF2 from a closely related species (e.g., another ungulate or another primate) may be designed to amplify within a cDNA sample nucleic acid sequences encoding IGF2. Table 1 (below) shows the percent sequence identity at both the protein and DNA level between different mammalian forms of IGF2.

TABLE 1
Percent Sequence Identity Between Different Mammalian Forms of IGF2

| | % | |
|---|---|---|
| Species | Identity Protein | % Identity DNA |
| Bos taurus vs. Homo sapiens | 84.8 | 86.5 |
| Bos taurus vs. Pan troglodytes | 68.0 | 74.2 |
| Bos taurus vs. Canis lupus familiaris | 84.4 | 88.5 |
| Bos taurus vs. Mus musculus | 81.5 | 83.1 |
| Bos taurus vs. Rattus norvegicus | 82.6 | 83.5 |

*Sequences and sequence alignment software were downloaded from NCBI website.

The level of IGF2 expression may also be determined by measuring the level of IGF2 protein in a mammalian cell or cell line. Methods of determining the level of IGF2 protein expression in a cell or cell line include, but are not limited to, Western blot, microscopic imaging using IGF2-specific antibodies tagged with fluorescent molecules, ELISA, and fluorescence-assisted cell sorting (FACS). In such methods, the level of expression of IGF2 protein may be normalized to the level of expression of a control protein in the cell (e.g., β-actin).

Antibodies specific for various forms of mammalian IGF2 are commercially available including antibodies specific for human (monoclonal and polyclonal human IGF2 antibodies from AbCam, Novus Biologicals, and LifeSpan BioSciences) and mouse IGF2 (AbCam). Due to the high degree of conservation between mammalian forms of IGF2 protein, polyclonal antibodies for human IGF2 have been shown to cross-react with other mammalian forms of IGF2 (e.g., polyclonal anti-human IGF2 antibody from Cell Sciences binds to chicken, human, porcine, and rat IGF2). Methods for the generation of further polyclonal and monoclonal antibodies or for the generation of polyclonal and monoclonal antibodies to other mammalian forms of IGF2 protein are also known in the art (e.g., Kohler et al., Nature 256:495-497, 1975; Campbell, “Monoclonal Antibody

Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985; Huse et al., Science, 246:1275-1281, 1989; and Kohler et al., Eur. J. Immunol, 6:511-519, 1976).

### Measurement of Mammalian p57 Expression Levels

p57 expression levels may be measured by determining the quantity of mRNA encoding p57 protein in a mammalian cell or cell line or by determining the quantity of p57 protein expressed in a mammalian cell or cell line.

The level of mRNA encoding p57 may be measured using PCR-based techniques, including real-time RT-PCR and RT-PCR. The specific primers used for each mammalian species may be designed from the available cDNA sequences for p57 from different mammalian species. The available p57 cDNA sequences include, but are not limited to, human (NCBI Accession Nos. NM_{—}001122631, NM_{—}001122630, NM_{—}000076, BC039188, and EU570054), rat (NCBI Accession Nos. NM_{—}001033757, NM_{—}001033758, NM_{—}182735, and BC098646), mouse (NCBI Accession No. NM_{—}009876), bovine (NCBI Accession Nos. BC123620 and NM_{—}001077903), and monkey p57 (NCBI Accession No. XM_{—}001117302). Primer sequences used to amplify and quantitate an mRNA encoding p57 in a mammalian cell may be designed using publicly available software on the NCBI website and software available on commercial websites (e.g., Invitrogen and Biosearch Technologies websites). An example of a set of specific primers used to amplify bovine p57 mRNA is described in the Examples. The level of p57 mRNA expression may also be normalized to the level of expression of a control gene in the cell (e.g., β-actin).

The cDNA sequence of p57 for mammalian species not yet cloned may be obtained using standard techniques in molecular biology. For example, due to the high degree of conservation of the protein sequence of p57 between mammalian species, degenerative primers based on the cDNA or protein sequence of p57 from a closely related species (e.g., another ungulate or another primate) may be designed to amplify within a cDNA sample nucleic acid sequences encoding p57. Table 2 (below) shows the percent sequence identity at both the protein and DNA level between different mammalian forms of p57.

TABLE 2
Percent Sequence Identity Between Different Mammalian Forms of p57

| Species | % identity protein | % identity DNA |
|---|---|---|
| Bos taurus vs. Homo sapiens | 70.2 | 74.6 |
| Bos taurus vs. Danio rerio | 39.9 | 46.0 |
| Bos taurus vs. Mus musculus | 66.3 | 76.1 |
| Bos taurus vs. Rattus norvegicus | 69.5 | 78.1 |

*Sequences and sequence alignment software were downloaded from NCBI website.

The level of p57 expression may also be determined by measuring the level of p57 protein in a mammalian cell or cell line. Methods of determining the level of p57 protein expression in a cell or cell line include, but are not limited to, Western blot, microscopic imaging using p57-specific antibodies tagged with fluorescent molecules, ELISA, and FACS. In such methods, the level of expression of p57 may be normalized to the level of expression of a control protein in the cell (e.g., β-actin).

Antibodies specific for various forms of mammalian p57 are commercially available including human (Abeam and Santa Cruz). Due to the high level of conservation between mammalian forms of p57 protein, polyclonal antibodies for human p57 protein have been shown to cross-react with other mammalian forms of p57 protein (e.g., polyclonal anti-p57 antibody from Abeam binds to human, mouse, and cow p57 protein). Methods for the generation of further polyclonal and monoclonal antibodies or for the generation of polyclonal and monoclonal antibodies to other mammalian forms of p57 protein are also known in the art (described above).

### Calculation of Mammalian IGF2/p57 Expression Ratio

The ratio of expression of IGF2/p57 levels may be measured by comparing the expression of IGF2 and p57 mRNA levels in a mammalian cell or cell line, or by comparing the IGF2 and p57 protein levels in a mammalian cell or cell line.

The methods for determining the level of expression of IGF2 and p57 mRNA in a mammalian cell or cell sample is described above. In one example, the level of IGF2 and p57 mRNA may first be normalized to the level of expression of a control gene in the cell (e.g., β-actin), and the normalized expression levels of IGF2 and p57 subsequently used to calculate the IGF2/p57 expression ratio.

An example of one PCR-based technique for measuring and determining the IGF2/p57 ratio is the method of real time RT-PCR. In this method, the RNA extracted from a mammalian cell or cell line is treated with reverse transcriptase to first generate a cDNA sample. The resulting cDNA sample is used to perform real time PCR using at least three sets of primers: a set of primers to amplify IGF2 cDNA, a set of primers to amplify p57 cDNA, and a set of primers to amplify a standard curve control. The data derived from the real time PCR reaction may be utilized to determine the ratio of IGF2/p57 expression in the mammalian cell or cell line. A description of the use of real time RT-PCR to determine the IGF2/p57 ratio in bovine cells is provided in the Examples.

### Determination of Allelic Expression of NNAT

Allelic expression (mono-allelic or bi-allelic expression) of a mammalian NNAT gene in a mammalian cell or cell line may also be determined by measuring the level of mRNAs encoding NNAT protein or by measuring the NNAT protein level in a mammalian cell or cell line.

The level of mRNAs encoding mammalian NNAT protein may be measured using a variety of PCR-based techniques, including real time RT-PCR or RT-PCR. The specific primers used for each mammalian species may be designed from the available cDNA sequences for NNAT from different mammalian species. The available NNAT cDNA sequences include, but are not limited to, human (NCBI Accession Nos. NM-_{—}005386 and NM_{—}181689), bovine (NCBI Accession Nos. NM_{—}178323 and BC103128), mouse (NCBI Accession No. BC036984), rat (NCBI Accession No. BC127473), and porcine NNAT (NCBI Accession No. NM_{—}001122990). Primer sequences used to amplify and quantitate the mRNA encoding NNAT may be designed using publicly available software on the NCBI website or software available on other commercial websites (e.g., Invitrogen and Biosearch Technologies websites). An example of specific pair of primers used to amplify bovine NNAT mRNA are described in the Examples. The primers may be specifically designed to target a portion of the cDNA sequence that contains a polymorphic sequence (e.g., a specific sequence within the cDNA molecules that differs between the two alleles of the gene).

In one example, the level of expression of NNAT (mono-allelic or bi-allelic expression) may be determined by performing real time RT-PCR using a cDNA sample prepared from an experimental mammalian cell or cell line and a control cell (e.g., a cell with established bi-allelic or mono-allelic expression of a NNAT gene). An example of a control cell with bi-allelic expression of the NNAT gene is an adult germ cell prior to gametogenesis, while a control cell with mono-allelic expression of the NNAT gene is an uncloned fetal fibroblast or germ cell following gametogenesis. Each real time RT-PCR reaction may include a set of primers to amplify the mammalian NNAT gene and a set of primers to amplify a control gene, such as β-actin (to generate a standard curve). By comparing the level of NNAT expression in individual samples, a standard level of expression may be established to define mono-allelic NNAT expression (e.g., below a specific value of NNAT expression) and bi-allelic NNAT expression (e.g., above a specific value of NNAT expression). Determination of mono-allelic or bi-allelic NNAT expression may also be determined by comparing the level of NNAT expression in a tested mammalian cell or cell line with that of a control cell line (e.g., a cell line that has established bi-allelic NNAT expression or mono-allelic NNAT expression).

Mono-allelic and bi-allelic expression of NNAT may also be determined by measuring the presence of allele-specific polymorphic sequences in a cDNA sample following RT-PCR. Polymorphic sequences within a mammalian species may be identified by performing PCR to generate cDNA encoding the NNAT sequence. The cDNA may be sequenced (e.g., Sanger-based methods) and polymorphic sequences present in each allele of the mammalian NNAT gene identified. For example, the identification and measurement of the polymorphic sequences present in two different alleles of the bovine NNAT gene are described in the Examples. Once polymorphic sequences have been identified for the different alleles of a mammalian NNAT gene, RT-PCR may be used to amplify a region of the cDNA sequence that contains the polymorphic sequences, and the resulting PCR products sequenced to determine whether both alleles of the NNAT gene are expressed in the mammal.

Mono-allelic and bi-allelic expression of NNAT may also be determined using in situ hybridization using fluorescent probes (i.e., FISH) specific for the NNAT gene. The probes used for hybridization may specifically recognize the polymorphic sequences unique to each allele of the mammalian NNAT gene. The probes used for hybridization may also recognize sequences shared between both alleles of the NNAT gene. Hybridization of the probes to genomic DNA may reveal whether the chromatin encoding one or both alleles of the NNAT gene is highly condensed. Mono-allelic expression of the NNAT gene may be evident when one copy of the chromosomal region encoding NNAT is highly condensed, while bi-allelic expression of the NNAT gene may be evident when both copies of the chromosomal region encoding the NNAT gene are highly condensed. FISH may also be used to detect whether the NNAT gene at a genomic locus is replicated in a synchronous or asynchronous manner. Synchronous replication of a gene at a genomic locus indicates an imprinted gene (e.g., a mono-allelically expressed gene), while asynchronous replication of a gene at a genomic locus indicates a bi-allelically expressed gene.

The relative level of NNAT protein may also measured to determine if the mammalian cell or cell line has mono-allelic or bi-allelic expression of the NNAT gene. Methods of determining the level of NNAT protein expression in a mammalian cell or cell line include, but are not limited to, Western blot, microscopic imaging using NNAT-specific antibodies tagged with fluorescent molecules, ELISA, and FACS. In such methods, the level of expression of NNAT protein may be normalized to the level of expression of a control protein in the cell (e.g., β-actin). The level of NNAT protein expression in a cell may be compared to the level of NNAT protein expression in a control cell (e.g., a cell that has mono-allelic or bi-allelic expression of the NNAT gene). As discussed above, mono-allelic NNAT protein expression may be defined as falling below a specific threshold value of NNAT protein expression, while bi-allelic NNAT protein expression may be defined as exceeding a specific value of NNAT protein expression. The specific values of NNAT protein expression used to define mono-allelic or bi-allelic protein expression may be normalized values, e.g., may be represented as the fold-expression level of NNAT protein in a mammalian cell or cell line relative to the expression level of a control protein (e.g., β-actin).

Antibodies specific for various forms of mammalian NNAT are commercially available including anti-human NNAT (Santa Cruz Biotechnology). Due to the high level of conservation between mammalian forms of NNAT protein, polyclonal antibodies for human NNAT have been shown to cross-react with other mammalian forms of NNAT (e.g., polyclonal anti-NNAT antibody from Santa Cruz Biotechnology binds to human, mouse, rat, and cow NNAT). Methods for the generation of further polyclonal and monoclonal antibodies or for the generation of polyclonal and monoclonal antibodies to other mammalian forms of NNAT protein are also known in the art (described above). Table 3 (below) shows the percent sequence identity at both the protein and DNA level between different mammalian forms of NNAT.

TABLE 3
Percent Sequence Identity Between Different Mammalian Forms of NNAT

| | % identity | |
|---|---|---|
| Species | protein | % identity DNA |
| Homo sapiens vs. Bos taurus | 61.0 | 88.0 |
| Homo sapiens vs. Pan troglodytes | 100.0 | 100.0 |
| Homo sapiens vs. Canis lupus familiaris | 100.0 | 96.7 |
| Homo sapiens vs. Mus musculus | 98.8 | 96.7 |
| Homo sapiens vs. Rattus norvegicus | 97.5 | 96.3 |

*Sequences and sequence alignment software were downloaded from NCBI website.

### Cloning Procedures

The methods of the invention provide for the identification of mammalian cells or cell lines that may be used successfully in cloning procedures. Several cloning procedures are known in the art, including, but not limited to, transfer of a donor cell and cell fusion (e.g., permeabilized cell transfer). Cloning procedures may also include the transfer of DNA (e.g., heterologous and/or homologous DNA) to the donor cell or oocyte before or after cell fusion. We have previously disclosed a variety of methods for cloning ungulates that may be used to clone mammals (see, e.g., U.S. Patent Application Publication No. 2002-0046722 and PCT Publication No. WO02/051997). In some of these methods, a permeabilized cell, prior to fusion or insertion into an enucleated oocyte, is incubated with a reprogramming media (e.g., a cell extract) to allow the addition or removal of factors from the cell, and the plasma membrane of the permeabilized cell is then resealed to enclose the desired factors and restore the membrane integrity of the cell. Other methods for the production of cloned mammals (e.g., ungulates) are known in the art, for example, in U.S. Pat. No. 5,995,577, assigned to University of Massachusetts, and in PCT Publication Nos. WO95/16670; WO96/07732; WO97/0669; and WO97/0668 (collectively, “the Roslin methods”). All of these patents are incorporated by reference herein in their entirety. These techniques are not limited to use for the production of transgenic bovines; the above techniques may be used for embryo cloning of other non-human mammals. Further, if desired, serial cloning may be carried out, in which the steps of any one or more of these methods may be repeated one or more times or different methods (e.g., reprogramming methods) may be performed sequentially to result in greater viability of the cloned fetuses.

Following embryo cloning, desired mammals may be produced by mating the mammals or by further gene targeting, genetic modification, or reprogramming as described below.

Transfer of a donor cell is one known cloning procedure that may be used to generate cloned cells or cloned offspring. Methods for the transfer of a donor cell (e.g., a permeabilized cell) are known in the art and are described, for example, in U.S. Pat. Nos. 7,414,170; 7,420,099; and 7,429,690 (each of which is incorporated herein by reference). A donor cell is preferably fused with or inserted into a recipient enucleated oocyte. Methods for the preparation of an enucleated oocyte are also known in the art, e.g., Liu et al., Mol. Reprod. Dev. 49:298-307, 1998; and Presicce et al., Mol. Reprod. Dev. 38:380-385, 1994. Prior to transfer, the donor cell may be genetically modified by gene targeting or introduction of nucleic acids (as described below).

DNA may be introduced into the donor cell (e.g., a permeabilized cell), oocyte, or embryo prior to or following any of the above cloning procedures. The transferred DNA may contain heterologous and/or homologous DNA sequences. The DNA may be a part of fragmented chromosome (e.g., a human chromosome fragment) or a mammalian artificial chromosome (e.g., a mammalian artificial chromosome that contains one or more homologous or heterologous transgenes). DNA may be introduced into a recipient oocyte, embryo, or donor cell by microinjection or lipofusion prior to or after one or more cloning steps (e.g., cell fusion or insertion of a donor cell into a recipient oocyte). DNA may also be introduced into other recipient cells, such as embryonic stem cells, adult stem cells, undifferentiated cells, undifferentiated cells, induced pluripotent stem cells, a cell derived from an embryo or blastocyst, or any cell described herein. Methods for the introduction of DNA are known in the art and are described, for example, in U.S. Pat. Nos. 4,994,384 and 5,945,577 (herein incorporated by reference); Summers et al., Biophys J. 71(6):3199-206, 1996; Nabekura et al., Pharm Res. 13(7):1069-72, 1996; Walter et al., Biophys J. 66(2 Pt 1):366-376, 1994; Yang et al., Biosci Rep.13(3):143-157, 1993; Walter et al., Biochemistry 6:32(13):3271-3281, 1993; and Collas et al., J. Cell Sci. 109, 1275:1283, 1996.

Reprogramming in Cloning Procedures

Mammalian cells used for cloning procedures may be reprogrammed (e.g., alteration in the expression of one or more genes in the cell) without requiring the isolation of nuclei from the cells. In reprogramming methods, cells may be permeabilized and then incubated in a reprogramming media (e.g., an extract from an interphase or mitotic cell) under conditions that allow the exchange of factors between the media (e.g., a cell extract) and the cells. If an interphase media is used, the nuclei in the cells remain membrane-bounded; if a mitotic media is used, nuclear envelope breakdown and chromatin condensation may occur. After the cells are reprogrammed by incubation in this media, the plasma membrane is preferably resealed, forming an intact reprogrammed cell that contains desired factors from the media and has altered expression in one or more of its genes. Methods for reprogramming cells are known in the art and are described, for example in U.S. Pat. No. 7,253,334 and U.S. Patent Application Publication No. 2006/0212952 (herein incorporated by reference). Reprogrammed cells may be fused with or inserted into recipient enucleated oocytes to yield embryos, which may be implanted in a maternal host to produce offspring. For the production of a mammal expressing a transgene (e.g., an antibody), cells may be genetically modified before, during, or after reprogramming by the insertion of a nucleic acid encoding the transgene. Cells from the initial cloned fetus or cloned offspring may be used in one or more additional cloning steps or may be frozen to form a cell line to be used as a source of donor cells for the generation of additional cloned mammals. Reprogrammed cells may also be cultured to generate cells for regenerative cell therapy.

Methods for Breeding Ungulates and Serial Cloning

In preferred embodiments of any of the above cloning methods, a cloned mammal may be mated with another mammal to produce an embryo, fetus, or live offspring with two or more genetic modifications (e.g., mutation or disruption of an endogenous gene, addition of a nucleic acid, or addition of an artificial chromosome). One or more cells may then be isolated from the embryo, fetus, or offspring, and one or more additional genetic modifications introduced into the isolated cell(s).

Gene Targeting

As described above, a donor cell may be genetically modified prior to a first or subsequent cloning step. The genetic modification of a donor cell may include inactivation, removal, or modification of a gene; upregulation of a gene; gene replacement; or transgene replacement at a predetermined locus. Examples of non-human genes that may be targeted resulting in their inactivation, removal, or modification are genes encoding antigens which are xenoreactive to humans (e.g., α-1,3 galactosyltransferase); genes in the PrP locus responsible for the production of the prion protein and its normal counterpart in non-human mammals; genes which in humans are responsible for genetic disease and which in modified, inactivated, or deleted form could provide a model of that disease in mammals (e.g., the cystic fibrosis transmembrane conductance regulator gene); genes responsible for substances which provoke food intolerance or allergy; genes responsible for the presence of particular carbohydrate residues on glycoproteins (e.g., the cytidine monophospho-N-acetyl neuraminic acid hydroxylase gene in non-human animals); and genes than can improve disease resistance, growth rate, milk/meat production, and tolerance to adverse environmental conditions.

Replacement of genes may also be performed. Genes that may be replaced include genes responsible for the production of blood constituents (e.g., serum albumin), genes responsible for substances that provoke food intolerance or allergy, and mutant forms of genes that cause disease.

Introduction of Nucleic Acids

If desired, nucleic acid molecules encoding a desired polypeptide may be inserted into an endogenous gene as part of the cloning process. For example, genes encoding a wild-type version of a diseased gene may be introduced into a cell. Preferably, human artificial chromosomes are used for this purpose. Following introduction of an artificial chromosome, the cell line (e.g., a fetal fibroblast) may be used as a donor cell for further gene targeting. As an alternative to the use of a human artificial chromosome, polynucleotides encoding genes of interest may also be introduced using a YAC vector, BAC vector, or cosmid vector. Such vectors may be introduced into cells (e.g., fetal fibroblasts cells) using known methods, such as electroporation, lipofection, fusion with a yeast spheroplast comprising a YAC vector, and the like. Desirably, vectors containing genes of interest may be targeted to the endogenous corresponding gene loci of the cells (e.g., fetal fibroblasts), resulting in the simultaneous introduction of the gene of interest and the mutation of the endogenous gene.

Integration of a nucleic acid encoding a gene of interest may also be carried out as described in the patents by Lonberg et al. (U.S. Pat. Nos. 5,545,806, 5,569,825, 5,625,126, 5,633,425, 5,661,016, 5,750,172, 5,770,429, 5,789,650, 5,814,318 5,874,299, 5,877,397, and 6,300,129, each of which is hereby incorporated by reference). In the “knock-in” construct used for the insertion of gene of interest into a chromosome of a host mammal, one or more genes and an antibiotic resistance gene may be operably-linked to a promoter which is active in the cell type transfected with the construct. For example, a constitutively active, inducible, or tissue-specific promoter may be used to activate transcription of the integrated antibiotic resistance gene, allowing transfected cells to be selected based on their resulting antibiotic resistance. Alternatively, a knock-in construct in which the knock-in cassette containing the gene(s) of interest and the antibiotic resistance gene is not operably linked to a promoter may be used. In this case, cells in which the knock-in cassette integrates downstream of an endogenous promoter may be selected based on the resulting expression of the antibiotic resistance marker under the control of the endogenous promoter. These selected cells may be used in the embryo cloning procedures described herein to generate a transgenic mammal containing a gene of interest integrated into a host chromosome. Alternatively, a mammal containing exogenous genes of interest may be mated with a mammal in which the endogenous gene is inactivated.

### Regenerative Cell Procedures

The invention also provides a cell or cell line for use in regenerative cell procedures. In these procedures, a target cell is modified to effect a change to an undifferentiated cell, adult stem cell, or ES cell phenotype and/or to alter the gene expression profile to that of an undifferentiated cell, an adult stem cell, or an ES cell. The resulting cell or cell line, demonstrating an undifferentiated, adult stem cell, or embryonic stem cell gene expression or phenotype, may be differentiated to a specific cell type for research or for administration to a patient for therapy. In addition, a cell or cell line demonstrating an undifferentiated, adult stem cell, or embryonic stem cell gene expression or phenotype may be used in any of the above cloning procedures to generate a viable embryo or viable offspring.

In another example, regenerative cell procedures provide for the culture of an undifferentiated cell, embryonic stem cell, or adult stem cell, and the subsequent treatment with agents and bioactive factors to elicit differentiation into a desired cell type. A common disadvantage of the culture of embryonic stem cells, adult stem cells, or undifferentiated cells is that the resulting culture comprises a heterogeneous population of cells with varied gene expression (e.g., heterogenous expression of imprinted genes) (Humpherys et al., Science 293:95-97, 2001). The heterogeneity in imprinted gene expression is thought to negatively affect the use of the cultured cells for further differentiation, research, or their use in cloning and therapeutic methods.

The present invention provides methods for identifying a cell or cell line that may be used in any of the above regenerative cell techniques. The invention provides methods for identifying a cell or cell lines that have decreased heterogeneity in imprinted gene expression and therefore allow for improved results in regenerative cell procedures. Examples of these procedures are described below. The regenerative cell procedures as described below may be used with any of the mammalian cells described herein, including, but not limited to, embryonic stem cells, adult stem cells, undifferentiated cells, cells from an embryo, or cells from a blastocyst.

Differentiation of Genetically-Stable ES, Adult Stem Cell, and Undifferentiated Cells

The methods of the invention provide a genetically-stable (e.g., decreased heterogeneity in imprinted gene expression) population of ES cells, adult ES cells, undifferentiated cells, or cells having the gene expression or cellular phenotype of an embryonic stem cell, an adult stem cell or undifferentiated cell that may be further differentiated to a specific mammalian cell type. For example, 10 μM all-trans-retinoic acid may be used to differentiate cells to a neuronal phenotype; culture for 21 days in 10 μM all-trans-retinoic acid, followed by culture in the presence of dexamethasone, insulin, and indomethacin may be used to differentiate cells to an adipocyte phenotype; culture for 21 days in dexamethasone, β-glycerophosphate, and L-ascorbate-2-phosphate may be used to differentiate cells to an osteogenic phenotype; and culture on methylcellulose may be used to differentiate cells to an endothelial cell phenotype.

In each of the above methods, the ES cells, adult stem cells, undifferentiated cells, or cells having the gene expression or phenotype of an ES cell, an adult stem cell, or an undifferentiated cell may be genetically modified prior to use in differentiation procedures. Genetic modification of these cells may include the replacement of a diseased copy of a gene with a wild-type copy of the gene, expression of a xenogenous gene (e.g., integrated into the genome or expressed from a mammalian artificial chromosome), or deletion or inactivation of an endogenous gene (e.g., one or both alleles of the gene). Methods for the genetic modification of a target cell are described above.

Reprogrammed Cells

Cell reprogramming may also be used in regenerative cell procedures. As described above cell reprogramming may involve the permeabilization of a target cell and incubation of the cell in an cell extract under conditions that allow the exchange of factors between the extract and the cells. Incubation of cells in extracts derived from ES cells, adult stem cells, or undifferentiated cells may be used to provide cells useful for regenerative cell therapy or for subsequent differentiation to a cell type of interest (as described above).

Cellular reprogramming may also be used to generate a mammalian cell suitable for cloning. For example, a differentiated target cell may be permeabilized and incubated with an extract from an oocyte (e.g., a metaphase II oocyte), an ES cell, an adult stem cell, or an undifferentiated cell, and the resulting cell may be propagated (e.g., at least 2, 3, 4, 5, 6, 7, 8, 9, 10, 15, 20, 30, 40, 50, 60, 70, 80, 90, 100, 150, 200, 250, 300, 350, or 400 cell divisions or population doublings) in vitro. The expanded reprogrammed cell culture may, if desired, be further differentiated using growth factors. The present invention provides methods for identifying reprogrammed cells for propagation in cell culture or for use in differentiation techniques. The invention provides a method of identifying reprogrammed cells that have decreased heterogeneity in imprinted gene expression and are more likely to result in a successful outcome in cell replacement therapy or cloning procedures.

Induced Pluripotent Stem Cells

The methods of the invention may also be used to identify induced pluripotent stem (IPS) cells that may be used in further cloning and regenerative cell procedures. Induced pluripotent stem cells are generated by the introduction of three or four transgenes into the target cell (e.g., Oct 3/4, Sox2, Klf4, c-Myc, Nanog, and Lin2), usually through the use of viral vectors. The expression of the introduced transgenes results in altered gene expression in the target cell (i.e., the IPS cell) that results in a gene expression pattern similar to that observed in an undifferentiated cell, an adult stem cell, or an ES cell. Induced pluripotent stem cells may be differentiated to express the phenotype of various differentiated cell types in vitro (using, for example, the above-described methods).

Cultured ES cells demonstrate heterogeneity in the expression of imprinted genes, and such heterogeneity is thought to result in a decrease in the effectiveness of the cells (e.g., research or therapy). The invention provides methods for identifying IPS cells that demonstrate a reduced level of heterogeneity in the expression of imprinted genes and are therefore more likely to provide a successful therapeutic outcome or an improved cell culture for research and cloning procedures.

Selective Removal of Cells from Cell Cultures for Use in Cloning and Regenerative Cell Procedures

The invention further provides methods for selectively removing one or more cells from a cell population that are less likely to yield a viable embryo, viable offspring, or a genetically-stable regenerative cell line following cloning and regenerative cell procedures. As described above, cultures of ES cells, adult stem cells, undifferentiated cells, cells having ES cell-, adult stem cell-, or undifferentiated cell-like gene expression or phenotype, reprogrammed cells, and IPS cells are thought to have heterogeneity in the expression of imprinted genes, which results in the decreased efficiency of using these cells in cloning and regenerative cell procedures. The invention provides methods for selectively removing one or more cells from such cell population that have a reduced likelihood of generating a viable embryo, viable offspring, or a genetically-stable regenerative cell line. Cells to be selectively removed from these cultures include cells that have an IGF2/p57 expression ratio of greater than 2 (e.g., greater than 3, 4, 5, 6, 7, 8, 9, or 10) and/or have bi-allelic expression of NNAT. Any standard method may be used to selectively remove such cells.

For example, one or more mammalian cells may be selectively removed from a cell population by the method of fluorescence-assisted cell sorting (FACS). In this method, cells may be incubated with fluorescently-labeled antibodies specific for mammalian IGF2, p57, and NNAT. The viable cells may be sorted based on their fluorescence intensity. The fluorescence intensity score for each of the fluorophores attached to the individual IGF2, p57, and NNAT antibodies may be normalized to the fluorescence intensity measured in a control cell that demonstrates a wild-type IGF2/p57 expression ratio and/or mono-allelic NNAT (e.g., an uncloned fetal fibroblast cell).

Another method for selective cell removal using FACS utilizes fluorescently labeled oligonucleotide probes specific for each allele of the NNAT gene. Following hybridization of the probes, FACS is used to selectively remove the cells demonstrating the fluorescence intensity of a control cell having bi-allelic or mono-allelic expression of the NNAT gene or a specific threshold level of fluorescence intensity determined through experimentation (e.g., a specific value relative to the fluorescence intensity of a probe for a control gene in the cell).

Selective Isolation of Cells from Cell Cultures for Use in Cloning and Regenerative Cell Procedures

The invention further provides methods for generating genetically-stable single-cell derived colonies from a hetergenous cell line (e.g., an IPS cell line, a ES cell line, an adult stem cell line, a population of reprogrammed cells, a fetal fibroblast cell line, or a population of cells having undifferentiated cell-like gene expression or phenotype). The single-cell derived colonies may be used in cloning procedures or regenerative cell procedures to generate a viable embryo, viable offspring, or a genetically-stable regenerative cell line. Cells to be selectively isolated from these cultures include cells that have an IGF2/p57 expression ratio of less than 11 (e.g., less than or equal to 10, 9, 8, 7, 6, 5, 4, 3, 2, or 1) and/or have mono-allelic expression of NNAT. Any standard method may be used to selectively isolate such cells.

For example, one or more mammalian cells may be selectively isolated from a cell population by different techniques using FACS. For example, cells may be incubated with fluorescently-labeled antibodies specific for mammalian IGF2, p57, and NNAT. The viable cells may be sorted based on their fluorescence intensity. The fluorescence intensity score for each of the fluorophores attached to the individual IGF2, p57, and NNAT antibodies may be normalized to the fluorescence intensity measured in a control cell that demonstrates a wild-type IGF2/p57 expression ratio and/or mono-allelic NNAT (e.g., an uncloned fetal fibroblast cell). Another method for selective cell isolation using FACS utilizes fluorescently labeled oligonucleotide probes specific for each allele of the NNAT gene. Following hybridization of the probes, FACS is used to selectively isolate the cells demonstrating the fluorescence intensity of a control cell having bi-allelic or mono-allelic expression of the NNAT gene or a specific threshold level of fluorescence intensity determined through experimentation (e.g., a specific value relative to the fluorescence intensity of a probe for a control gene in the cell). Cells with an IGF2/p57 expression ratio of less than 11 (e.g, less than or equal to 10, 9, 8, 7, 6, 5, 4, 3, 2, or 1) and/or have mono-allelic NNAT expression may be selectively isolated using these techniques.

### Selection of Embryos for Implantation

The invention further provides methods for selecting a fertilized embryo for implantation into a maternal mammalian host. As described above, the lack of correct expression of imprinted genes has been correlated with a lack of efficiency in the generation of a viable offspring. The methods of the invention provide a means to identify embryos that have an imprinted gene expression profile that correlates with the successful generation of a viable offspring. In particular, embryos (e.g., both one-cell embryos and multicell embryos) which express an IGF2/p57 ratio of greater than 2 (e.g., greater than 3, 4, 5, 6, 7, 8, 9, or 10) and/or have mono-allelic expression of a NNAT gene are identified as having an increased likelihood of resulting in the successful production of an offspring, and should be chosen for implantation into a maternal host.

Selection of embryos having an IGF2/p57 ratio of greater than 2 (e.g., greater than 3, 4, 5, 6, 7, 8, 9, or 10) and/or have mono-allelic expression of a NNAT gene may be identified by incubation of the embryo in fluorescently-tagged antibodies specific for IGF2, p57, and/or NNAT. The resulting staining pattern may be compared to the staining observed in other control embryos (e.g., from the same mammalian species or a related mammalian species, or a control fetal fibroblast). In multicellular embyos, a few cells from the embryo may be removed and real time RT-PCR or PCR may be performed as described above to determine the expression levels of IGF2, p57, and/or NNAT. In addition, fluorescently labeled oligonucleotide probes specific for each allele of the NNAT gene may be used to determine the allelic expression of NNAT (e.g., mono-allelic or bi-allelic NNAT expression). The fluorescence pattern may be compared to the fluorescence in other control embryos (e.g., from the same mammalian species or a related mammalian species, or a control fetal fibroblast).

### Embryo Screening

The invention also provides methods for measuring the likelihood that an offspring will have altered imprinted gene expression. In these methods, a sample of cells (e.g., placental cells) are removed from the maternal host, and the expression level of IGF2, p57, and/or NNAT are determined using the above-described techniques. Embryos which express an IGF2/p57 ratio of less than 9 (e.g., less than 8, 7, 6, 5, 4, 3, 2, or 1) and/or have mono-allelic expression of a NNAT gene are likely to develop into a viable offspring having normal expression of imprinted genes and epigenetic programming. This method and the above methods may be used to select healthy mammalian clones or as a method of prenatal diagnosis, for example, for human patients (e.g., for selecting fertilized embryos for implantation by in vitro fertilization procedures).

While the approaches to be utilized in the invention have been described above, the techniques that are utilized are described in greater detail below. These examples are provided to illustrate the invention, and should not be construed as limiting. In particular, while these examples focus on bovines, the methods described may be used to produce and test any mammal, mammalian cell, or mammalian cell line.

### Examples

### Example 1

### Measurement and Statistical Analysis of the Expression Levels of Different Imprinted Genes in Bovine Fetuses

In order to identify imprinted genes that play an important role in the successful generation of a viable cloned embryo, a cloned offspring, or a genetically-stable regenerative cell line, we analyzed the correlation between the successful generation of a cloned bovine offspring (i.e., calving rate) and the expression levels of several imprinted genes (epigenetic markers) in bovine cell lines. In this study, we analyzed a panel of imprinted genes from several hundred cloned bovine fetal fibroblast cell lines (i.e., cells derived from a bovine fetus) from three different genetic breeds.

Of the 21 imprinted genes analyzed, we found that the expression of three imprinted genes (i.e., IGF2, p57, and NNAT) strongly correlated with the likelihood of generating a viable bovine offspring via cloning procedures. Specifically, we discovered that a disturbance in the balance of IGF2 and p57 gene expression levels (as indicated by the IGF2/p57 ratio) has an inverse correlation with the likelihood that use of the cell line or the cell in a cloning procedure will result in a viable offspring (i.e., “calving potential”). Similarly, we discovered that a loss of imprinting of the NNAT gene (i.e., a switch from mono-allelic expression to bi-allelic expression) in a cell or a cell line also inversely correlated with the likelihood that use of the cell or cell line in a cloning procedure would result in a viable offspring.

In the first analysis of the data, a cell line was indicated as clonable if it produced at least one calf following cloning procedures and a cell line was deemed unclonable when no calf was produced following cloning procedures. The data indicated that 85.7% of the cell lines with an increased IGF2/p57 ratio of 9.0 and 72.0% of cell lines with bi-allelic expression of NNAT were unclonable. Conversely, 86.9% of cell lines not having an IGF2/p57 ratio of 9.0 and bi-allelic NNAT expression were clonable (i.e., were shown to produce at least one viable offspring using cloning procedures).

Further statistical analysis of the data was performed by using 2% and 5% calving rates (i.e., 2% or greater than 2%, or 5% or greater than 5% of the implanted embryos resulted in a viable offspring following cloning procedures) as the cutoff value for defining a clonable versus unclonable cell line. We found that the predictive value of the IGF2/p57 ratio and bi-allelic expression of NNAT for the successful production of a viable offspring was more robust when used to predict whether a cell line would result in a 2% calving rate or 5% calving rate. When the 2% calving rate was used as the cutoff value, 95.2% and 72.0% of cell lines having an IGF2/p57 ratio of 9.0 and bi-allelic expression of NNAT, respectively, were unclonable. When the 5% calving rate was used as the cutoff value, 95.2% and 76.0% of cell lines having an IGF2/p57 ratio of 9.0 and bi-allelic expression of NNAT, respectively, were unclonable.

Further statistical analyses of these data are shown in FIGS. 1-3. FIG. 1 shows that 92.3% of cells (G0-G1) having an IGF2/p57 expression level of less than 9 result in the successful generation of a live offspring, whereas only 33.3% of cells (G0-G1) having an IGF/p57 expression level of greater than 9 result in the generation of a live offspring. FIG. 2 shows that 83.3% of cells (G0-G1) having mono-allelic expression of NNAT result in the generation of a live offspring, while only 27.8% of cells (G2) having bi-allelic expression of NNAT result in the generation of a live offspring.

The predictive effect of the combination of the IGF2/p57 ratio and the allelic expression of NNAT provides a heightened level of accuracy in predicting the success of a cloning procedure. FIG. 3 shows that 100% of cells (G0-G1) having an IGF2/p57 ratio of less than 9 and mono-allelic expression of NNAT resulted in the generation of a live offspring, while 50% of cells (G0-G1) having an IGF2/p57 ratio of greater than 9 and bi-allelic expression of NNAT resulted in the generation of a live offspring.

It should be noted that the predictive power of the IGF2/p57 ratio and the bi-allelic expression of NNAT is decreased in highly cloned cell lines (G2-G5). These results indicate that there may be additional errors that accumulate in multiply cloned cells and cell lines.

The identification of IGF2/p57 and NNAT as epigenetic markers for the prediction of the successful creation of a viable embryo, viable offspring, and genetically-stable cell lines was unexpected. For example, other imprinted genes, previously thought to be important for the survival and development of cloned embryos, such as, Lit1 and H19, were not identified as a relevant epigenetic marker for the prediction of the generation of a viable offspring.

### Example 2

### Measurement of the IGF2/p57 Ratio in Bovine Fetuses

To measure IGF2 and p57 expression, RNA was isolated from bovine fetal fibroblasts derived from 40-day fetuses. The fetuses used for measurement were either control fetuses (no previous cloning steps; G0) or fetuses that had previously undergone one, two, three, four, or five prior cloning steps (i.e., G1, G2, G3, G4, or G5). The cells were harvested, and the RNA was isolated from the fetal fibroblasts using Ambion Total RNA isolation kit according to the manufacturer's instructions.

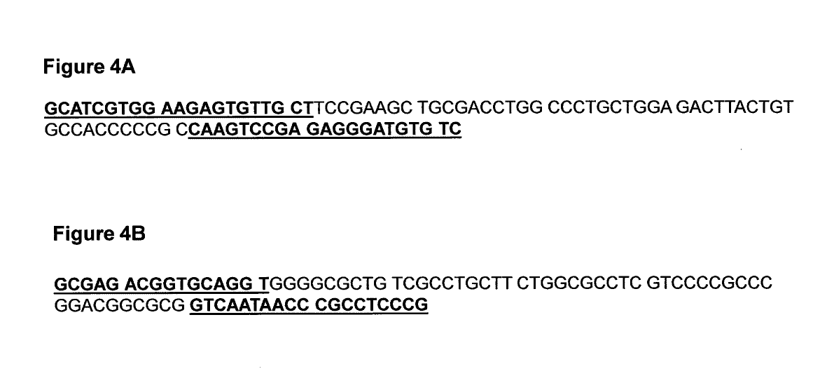

The isolated RNA from each sample was used for cDNA synthesis using Applied Biosystems High Capacity cDNA Synthesis Kit according to the manufacturer's instructions. Real Time PCR was performed to measure the resulting relative levels of IGF2 and p57 cDNA in the sample using an Applied Biosystems 7500 Fast Real-Time PCR machine. The primers used for IGF2 were 5′-GCATCGTGGAAGAG TGTTGCT-3′ (SEQ ID NO: 5) and 5′-CAAGTCCGAGAGGGATGTGTC-3′ (SEQ ID NO: 6). The primers used for p57 were 5′-GCGAGACGGTGCAGGT-3′ (SEQ ID NO: 7) and 5′-CTGAATAACCCGCCTGGGC-3′ (SEQ ID NO: 8). The amplified PCR products for bovine IGF2 and bovine p57 are shown in FIG. 4A and FIG. 4B, respectively.

The analysis of the data was performed using a standard curve method using G0 40-day fetal fibroblast cell line as a reference and β-actin as the endogenous reference gene. For each cell sample, the level of IGF2 and p57 expression was first normalized to β-actin, and then the normalized IGF2 expression level was compared to the normalized p57 expression level to determine the IGF2/p57 ratio.

### Example 3

### Measurement of the NNAT Allelic Expression in Bovine Fetuses

To measure NNAT expression, RNA was isolated from bovine fetal fibroblasts derived from 40-day fetuses. The fetuses used for measurement were either control fetuses (no previous cloning steps; G0) or fetuses that had previously undergone one, two, three, four, of five prior cloning steps (i.e., G1, G2, G3, G4, or G5). The cells were harvested, and the RNA was isolated from the fetal fibroblasts using Ambion Total RNA isolation kit according to the manufacturer's instructions.

The isolated RNA was used for cDNA synthesis using Applied Biosystems High Capacity cDNA Synthesis Kit according to the manufacturer's instructions. The NNAT coding region containing single nucleotide polymorphisms (SNPs) was amplified by PCR. Each PCR reaction contained: 2 μL cDNA, 5 μL 10× Taq polymerase buffer, 8 μL dNTPs mix (2 mM each), 1.5 μL forward (20 μM/μL) and 1.5 μL reverse primers (20 pM/μL), 0.5 U Ex Taq, and 31.5 μL dH₂O. The conditions for PCR used were one cycle of 85° C. for 3 minutes, 94° C. for 1 minutes, followed by 30 cycles of 98° C. for 10 seconds, 60° C. for 30 seconds, and 72° C. for 1 minute. The resulting RT-PCR products were DNA sequenced, and the allelic expression status of NNAT in each tested cell line was determined based on the chromatograms generated from DNA sequencing.

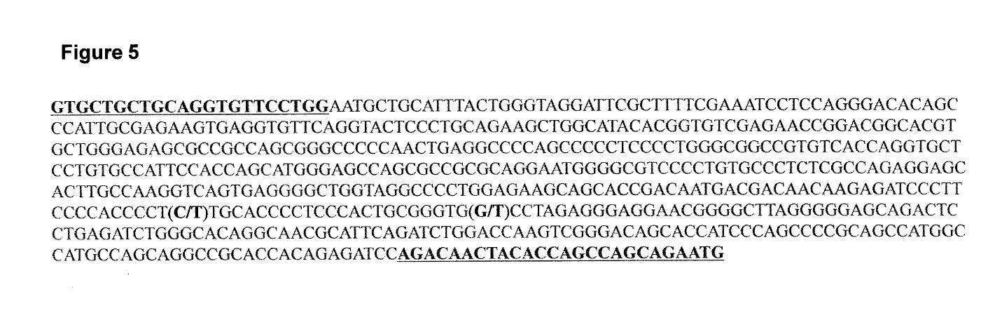

The primers used for NNAT were 5′-GTGCTGCTGCAGGTGTTCCTGG-3′ (SEQ ID NO: 9) and 5′-AGACAACTACACCAGCCAGCAGAATG-3′ (SEQ ID NO: 10). The amplified PCR products for both alleles of bovine NNAT are shown in FIG. 5. A DNA sequencing chromatograph from a sample having bi-allelic expression of NNAT indicates the expression of the polymorphic sequences in each allele of the bovine NNAT gene.

### Other Embodiments

All publications and patents cited in this specification are incorporated herein by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for identifying a mammalian cell for use in a cloning procedure or a regenerative cell procedure comprising the steps of:
a) measuring the expression level of insulin-like growth factor-2 (IGF2) in the cell;
b) measuring the expression level of p57 in the cell; and
c) determining the ratio of the expression level of IGF2 to p57 in the cell, wherein an IGF2 to p57 ratio of less than or equal to 9 identifies a cell for use in a cloning procedure or a regenerative cell procedure.

2. The method of claim 1, wherein said method identifies a mammalian cell having at least a 40% probability of resulting in the generation of a viable embryo, a viable offspring, or a genetically-stable regenerative cell culture.

3. The method of claim 1, wherein said cloning procedure or regenerative cell procedure comprises the steps of permeabilizing a cell and incubating the cell with a cell extract.

4. The method of claim 1, wherein said method results in the generation of a genetically-stable regenerative cell culture.

5. The method of claim 1, wherein said mammalian cell is an ungulate cell.

6. The method of claim 5, wherein said ungulate cell is a bovine, porcine, ovine, or caprine cell.

7. The method of claim 5, wherein said method is a regenerative cell procedure and said cell is a human cell.

8. A method for identifying a mammalian cell for use in a cloning procedure or a regenerative cell procedure comprising the step of determining whether the cell has mono-allelic expression of the neuronatin (NNAT) gene, wherein mono-allelic expression of NNAT identifies a cell for use in a cloning procedure or a regenerative cell procedure.

9. The method of claim 8, wherein said method identifies a mammalian cell having at least a 50% probability of resulting in the generation of a viable embryo, a viable offspring, or a genetically-stable regenerative cell culture.
